(19) United States
(12) Patent Application Publication
(10) Pub. No.: US 2024/0428911 A1
(43) Pub. Date: Dec. 26, 2024

(54) ARTIFICIAL INTELLIGENCE SYSTEMS AND METHODS FOR PREDICTING AND AVOIDING PHYSICAL AND PSYCHOLOGICAL DOWNTURNS ASSOCIATED WITH CHRONIC DISEASE

(71) Applicant: IBDAlly Health Corporation, Alpharetta, GA (US)

(72) Inventors: Scott G. Genone, Roswell, GA (US); Ricardo Diaz, Cocoa Beach, FL (US); Summerpal Kahlon, Canton, GA (US); Richard Alan Burton, Atlanta, GA (US)

(73) Assignee: IBDAlly Health Corporation, Alpharetta, GA (US)

(21) Appl. No.: 18/750,824

(22) Filed: Jun. 21, 2024

Related U.S. Application Data
(60) Provisional application No. 63/522,769, filed on Jun. 23, 2023.

(51) Int. Cl. G16H 20/00; G16H 40/20; G16H 50/20; G16H 50/30; G16H 50/70

(52) U.S. Cl. CPC G16H 20/00; G16H 40/20; G16H 50/20; G16H 50/30; G16H 50/70

(57) ABSTRACT

Artificial intelligence computer systems according to various embodiments predict and facilitate taking action to avoid potential physical and/or psychological downturns associated with a chronic disease, such as inflammatory bowel disease. The systems may be adapted to use a rules-based model and/or a machining-learning model to generate, for a particular patient, a predication of a future flair-up or relapse of a particular medical condition associated with the chronic disease and to automatically take one or more actions to prevent the predicted flair-up or relapse. The one or more actions may include, for example: (1) having one or more food items delivered to the particular patient; (2) scheduling an appointment for the particular patient to treat the chronic disease; and/or (3) scheduling transportation for the patient via a ride-sourcing or taxi service.

### CROSS-REFERENCE TO RELATED APPLICATIONS

This application claims the benefit of U.S. Provisional Patent Application Ser. No. 63/522,769, filed Jun. 23, 2023, the disclosure of which is hereby incorporated herein by reference in its entirety.

### BACKGROUND

Individuals with chronic diseases, such as inflammatory bowel disease (“IBD”) often face significant challenges in receiving the proper care for their conditions. For example, such individuals may have difficulty: (1) scheduling appointments that are crucial to their care; (2) identifying the best available providers for their care; (3) obtaining insurance pre-authorization for necessary drugs or procedures; (4) coordinating transportation to various medical facilities to obtain treatment; (5) coordinating healthy meals that are helpful in avoiding relapses or flare-ups in their condition; (6) arranging for mental health support to help cope with their condition; (7) adhering to suitable disease-management programs; (8) obtaining the best pricing on medications; (9) tracking medication delivery; (10) monitoring food triggers; (11) coordinating financial help through patient assistance programs; (12) identifying mistakes in billing associated with their condition; (13) storing and retaining documents related to their condition; and (14) keeping up to date on their condition and related medications, etc.

There are significant technical challenges associated with designing computer systems and related methods to help address the issues above. For example, various embodiments of such a computer system would preferably: (1) properly manage an individual's various consents to take action on the individual's behalf; (2) include sufficient data security to assure compliance with applicable data privacy laws, such as HIPPA; and (3) include advanced technology to facilitate immediate treatment of (or avoidance of) relapses or flare-ups in the condition.

### SUMMARY

A system, according to various embodiments, comprises: (1) a non-transitory computer-readable medium storing computer-executable instructions; and (2) a processing device communicatively coupled to the non-transitory computer-readable medium, where the processing device is configured to execute the instructions and thereby perform operations comprising: (1) receiving a set of patient event data, the set of patient event data including at least one respective instance of a respective patient event experienced by each respective patient of a plurality of patients; (2) receiving a first set of parameters, the first set of parameters including a respective set of one or more parameters for each respective patient of the plurality of patients that were associated with the respective patient immediately before or while the patient experienced the at least one instance of the respective patient event; (3) receiving, for a particular patient, a set of particular patient parameters; (4) processing the first set of parameters, the set of patient event data, and the set of particular patient parameters using at least one of a rules-based model or a machine-learning model to generate a prediction of a future patient event for the particular patient; (5) receiving, for each respective patient event, respective event mitigation data; (6) receiving, for each respective patient event, respective event outcome data; (7) processing the prediction of the future patient event, the respective event mitigation data, and the respective event outcome data to generate a future patient event mitigation recommendation; and (8) facilitating implementation of one or more mitigating actions defined by the future patient event mitigation recommendation.

A computer-implemented data processing method for improving the prediction and automated remediation of a future medical event, according to various embodiments, comprises: (1) receiving, by computing hardware, a set of patient parameters for a particular patient; (2) analyzing, by the computing hardware to produce a first data analysis result, the set of patient parameters using a machine-learning model trained with a set of patient parameter data derived from a plurality of patients, each of the plurality of patients having experienced a respective medical event from a set of medical events and having at least one respective parameter from the set of patient parameters; (3) generating, by the computing hardware based on the first data analysis result, a prediction as to an occurrence of the future medical event for the particular patient, the future medical event including at least one medical event from the set of medical events; (4) generating, by the computing hardware, a graphical user interface by configuring a display element that includes the prediction; and (5) providing, by the computing hardware, the graphical user interface for display on a user device.

A computer-implemented data processing method, according to particular embodiments, comprises: (1) obtaining, by computer hardware, one or more consents from an individual to facilitate: (a) treating a particular condition; (b) paying for one or more services related to a particular condition, or (c) advocating on behalf of a particular individual in relation to managing a particular condition; (2) securely storing, by computer hardware, data documenting the one or more consents in a consent management data structure; (3) receiving, by computer hardware, an indication that a particular consent is needed in order to execute a particular operation on behalf of the particular individual; and (4) in response to receiving the indication, returning, by computer hardware, data indicating whether the individual has provided the particular consent.

A computer-implemented data processing method, according to various embodiments, comprises: (1) training, by computer hardware, a machine learning model on patient data from a plurality of patients with a particular medical condition; (2) using, by computer hardware, a machine learning model to identify one or more risk factors associated with a relapse or flare-up in the particular medical condition; (3) receiving, by computer hardware, patient data for a particular patient; (4) using, by computer hardware, the one or more risk factors identified by the machine learning model along with the received patient data to assess the immediate or future risk of the particular patient suffering a relapse or flare-up in the particular medical condition; and (5) at least partially in response to determining that the risk of the particular patient suffering a flare-up or relapse in the immediate or near future exceeds a particular threshold, automatically facilitating, by computer hardware, action by a third party computing system to take action to prevent or treat a flare-up or relapse in the patient.

### BRIEF DESCRIPTION OF THE DRAWINGS

In the course of this description, reference will be made to the accompanying drawings, which are not necessarily drawn to scale, and wherein:

FIG. 1 depicts an example of a computing environment that can be used for: (1) securely managing consents to take action to assist an individual in managing a health-related condition and/or in managing payments for treatments related to managing such a condition; (2) managing and storing an individual's healthcare-related data while also assuring compliance with one or more privacy laws or standards, such as HIPPA; and (3) predicting relapses and or flare-ups in an individual's condition and, in response, automatically initiating an action via a third party computing system to prevent or treat the predicted relapse or flare-up;

FIG. 2 depicts an example of a process for managing patient consents;

FIG. 3 depicts an example of a process for predicting flare-ups or relapses in a patient's medical condition and automatically facilitating action by a third-party computer system to take action to prevent or treat the flare-up or relapse;

FIG. 4 depicts an example of a process for automatically analyzing medical invoices to identify billing errors;

FIG. 5 depicts an example of a system architecture that may be used in accordance with various aspects of the present disclosure; and

FIG. 6 depicts an example of a computing entity that may be used in accordance with various aspects of the present disclosure.

### DETAILED DESCRIPTION

### Overview and Technical Advantages

Various aspects of the present disclosure overcome many of the technical challenges mentioned above. In various embodiments, the computer system may do this by providing a secure user interface that preferably facilitates: (1) the central intake and secure storage of patient data; (2) the coordination of secure communication with the computer systems of a variety of product and service providers that provide services for individuals with a particular condition, where such service providers include, for example, healthcare providers (e.g., physical and mental healthcare providers), insurance providers, and pharmaceutical providers; and (3) the management of consents needed for the system to facilitate the coordinated provision of services between various providers. The system may also be configured to facilitate electronic communication with one or more non-medical service providers such as grocery delivery services, fresh food delivery services, online marketplaces (e.g., Amazon.com or Walmart.com) transportation service providers such as ride-sourcing services (e.g., Uber or Lyft), taxi services, or public transportation services.

One primary goal of the system is to allow an individual to manage the treatment of their condition and to obtain assistance with advocating for help with managing their condition through a single user interface. In a particular embodiment, the system collects a large amount of data regarding an individual through a plurality of different sources. These sources may include, for example: (1) manual input by the user; (2) the user's electronic medical records; (3) information gathered by one or more wearable devices (e.g., smart watches, step trackers): (4) medical peripheral devices such as one or more blood pressure measurement devices or electronic scales; and/or (5) information gathered by the individual's smartphone. The collected data may include, for example: (1) symptoms that the individual is currently experiencing; (2) information regarding the individual's state of mind; (3) the amount and type of exercise that the individual completes; (4) information regarding the medications that the individual has taken; (5) information regarding any physical therapy that the individual has completed; (6) dietary information (information regarding the specific food and liquids that the individual has consumed); (7) information regarding the individual's bowel movements; (8) information regarding the individual's menstrual cycle; (9) information from a peripheral device that analyzes the patient's blood or urine, etc.

In various embodiments, the system may also take a picture of the individual on a daily basis (e.g., a picture of the individual's face or one or more other body parts and store these images as part of the individuals electronic medical record). The system may also coordinate the intake of one or more pictures of the individual's bowel movements (e.g., taken on a daily basis). In particular embodiments, the system collects this information on an ongoing basis and stores this information in memory. This may allow the system to collect and maintain a detailed historical record of the patient's medical information that can be studied and mined using one or more rule-based and/or machine learning computer processing techniques to identify one or more trends over time that may be used to facilitate and enhance the patient's medical treatment.

In particular embodiments, the system may gather such data for a plurality of patients (e.g., a large number of patients, such as over about 100 or over about 1,000 patients) having the same condition. This may allow the system to use big data/machine learning techniques to identify one or more trends associated with the condition. For example, the system may identify one or more factors that are associated with the flare-up or relapse in the client's condition.

As a particular example, the system may determine that one or more of the following factors, alone or in combination, are commonly associated with flare-ups in a particular condition: (1) particular changes in a patient's weight; (2) one or more particular states of mind (e.g., on a particular day or over a particular span of time); (3) compliance or lack of compliance with a particular drug regimen; (4) compliance or a lack of compliance with a particular dietary program; (5) intake of particular foods or beverages (e.g., dairy products); (6) exposure to particular environments, such as smoky or otherwise polluted environments; (7) compliance or lack of compliance with a particular exercise program; (8) the shape or structure of the patient's bowel movements; and/or (9) specific properties of the patient's blood or urine, or other factors.

The system may make such determinations for the patient alone and/or for a group of patients with the same or similar condition. In particular embodiments, the system may determine a risk assessment indicator (such as a percentage) that indicates how frequently the identified one or more factors are actually associated with a flare-up or relapse in the patient's condition. For example, the system may determine that 50% of the time when a particular patient consumes dairy products and hasn't taken a particular medication within the past 24 hours, the patient sufferers a flair-up in their condition. As another example, the system may determine that, on average, 40% of the time when a particular individual within a particular group of individuals has been depressed for over a week, has consumed more than a particular threshold number of alcoholic beverages within that week, and has gained over a particular percentage of their body weight within that week, they suffer a flare-up or a relapse in their condition sometime within the following week (or within any other specified period of time).

In particular embodiments, the system may take action in response to determining that the risk of a particular individual suffering a flare-up or relapse exceeds a particular threshold value. For example, if the system determines that, at a particular time, a particular individual has over a 70% chance of suffering a relapse or flare-up in the following week, the system may decide to take one or more actions to prevent or reduce the severity of the predicted potential flare-up or relapse.

For example, the system may display a message to the individual indicating that they are at a particular risk of suffering a potential flare-up or relapse and provide instructions to the individual including measures that they may take to potentially avoid the flare-up or relapse. As a particular example, if the system determines that the individual is at risk of suffering a relapse or a flare-up because they haven't taken one or more particular medications, the system may automatically convey (e.g., display on a display screen) a message to the user indicating that they should take the particular medicine.

As an additional example, in response to determining that particular individual has over a threshold chance of suffering a relapse or flare-up in the following week, the system may initiate electronic communication with a third party computing system (e.g., via a suitable application programming interface (API)) and through that communication initiate an action that is intended to avoid or treat the anticipated relapse or flare-up. For example, the system may automatically order one or more days' worth of groceries to be delivered to the individual's home. In particular embodiments, before doing so, the system may have automatically contacted a dietary professional or accessed a database containing dietary information and used information identified from that step to select groceries that are intended to avoid or treat the anticipated relapse or flare-up. In such an example, the groceries to be delivered to the individual's home would include those selected groceries.

As a further example, in response to determining that the particular individual has over a threshold chance of suffering a relapse or flare-up in the following week, the system may automatically schedule an appointment (e.g., either an in person or virtual appointment), such as an appointment with an appropriate physician, mental health professional, physical therapist, and/or other service provider. In particular embodiments, the system may automatically select the service provider for the appointment based, for example, on one or more symptoms that the individual is anticipated to experience during that predicted flare-up or relapse. For example, the system may automatically search one or more reviews of a plurality of relevant service providers and, based at least in part on the reviews from other customers of the service provider, select a provider that is indicated by the reviews to have experience treating flare-ups or relapses of the particular condition and/or symptoms of such flare-ups or relapses.

In various embodiments, the system may also coordinate transportation for the individual (e.g., through a particular ride sourcing service) to the relevant appointment. For example, the system may automatically coordinate transportation for the individual to and/or from an appointment with a particular medical provider via a suitable ride coordination service such as Uber or Lyft.

In various embodiments, when making such scheduling such appointments, the system may be configured to automatically add one or more entries to one or more electronic calendars associated with the individual to indicate when the appointment will occur and/or when the scheduled transportation to the appointment will happen. Similarly, when scheduling the delivery of items such as groceries or other items to the individual, the system may also automatically add one or more entries to one or more electronic calendars associated with the individual to indicate that such items are to be delivered and/or when such items are to be delivered.

In particular embodiments, the system may coordinate the delivery of one or more kits of items that have been assembled to treat the predicted type of flare-up or relapse. For example, the kit may include two or more particular medical supplies that have been assembled together into a kit for purposes of treating such flare-ups or relapses.

Furthermore, in particular embodiments, the system may be adapted to, in response to predicting that an individual may suffer a particular flare-up or relapse, automatically notify one or more physicians or other individuals associated with the particular individual. Such other individuals may include, for example, the individual's spouse, parent(s), or other caregivers or patient advocates.

The system may be further adapted to, in response to predicting that an individual may suffer a particular flare-up or relapse, automatically initiate a request for one or more insurance pre-authorizations for one or more healthcare appointments and/or procedures needed to address the predicted flare-up or relapse. This may, for example, eliminate delays in the individual obtaining treatment if they suffer the anticipated flare-up or relapse. In various embodiments, the system may obtain suitable pre-authorizations for particular appointments before scheduling such appointments and/or corresponding transportation as discussed above.

In various embodiments, the system may be further adapted to assist an individual in managing various financial aspects of managing their condition. For example, the system may be adapted to automatically assist an individual in identifying and obtaining one or more available discounts on one or more drugs or other items or services associated with the individual's condition. This may include, for example, identifying one or more coupons associated with the drugs, or identifying one or more discounts or patient assistance programs through which the one or more drugs (or other items or services) are available at a discounted price.

As another example, the system may be adapted to automatically review one or more financial transactions associated with one or more goods or services associated with treating the individual's condition to identify any potential billing errors associated with the one or more financial transactions. In response to identifying the one or more billing errors, the system may automatically initiate one or more actions to resolve those errors. For example, the system may automatically generate an electronic communication to an entity associated with the relevant financial transaction, where the electronic communication identifies the financial transaction, the patient, and/or the identified billing error. The system may, alternatively or in addition, generate such a communication to the individual so that the individual may be aware of the billing error and/or take action to correct the billing error.

In various embodiments, the system may include additional features such as: (1) assisting the individual with complying with a suitable disease management program; (2) tracking the delivery of drugs or other items to the individual; (3) providing a suitable electronic repository for storing electronic files or other items related to the individual's condition; and (4) keeping the individual up to date on both their condition and related medications to treat their condition.

In particular embodiments, in order to facilitate various aspects of the system described above, the system may include a consent management system for managing obtaining relevant consents from the individual and for making sure that the system has the necessary consents in place before taking corresponding action on behalf of the individual. Such consents may include for example: (1) consent for the system or an entity associated with the system to advocate on the individual's behalf with the individual's insurance company; (2) consent for the system to share the patient's medical data with one or more medical providers or other individuals such as one or more of the individual's relatives, caregivers, or patient advocates; (3) consent for the system to pay for one or more services or items from one or more payment sources specified by the individual—such payment sources may include, for example, one or more credit cards, one or more bank accounts, or other funding sources; or (4) consent for the system to use one or more logins for one or more apps or programs associated with the individual to order goods or services through those apps or programs (for example, this consent may include authorization for the system to order items from the individual's Amazon.com account or to order transportation through the individual's Uber account).

It should be understood, based on the current disclosure, that many technical challenges exist with managing such consents and assuring that the consents stay secure and are only used for the intended purposes. From a technical standpoint, there are also challenges associated with assuring that each individual's data is maintained in a secure storage location in compliance with applicable privacy laws such as HIPAA.

### MORE DETAILED DESCRIPTION

### Example Computing Environment

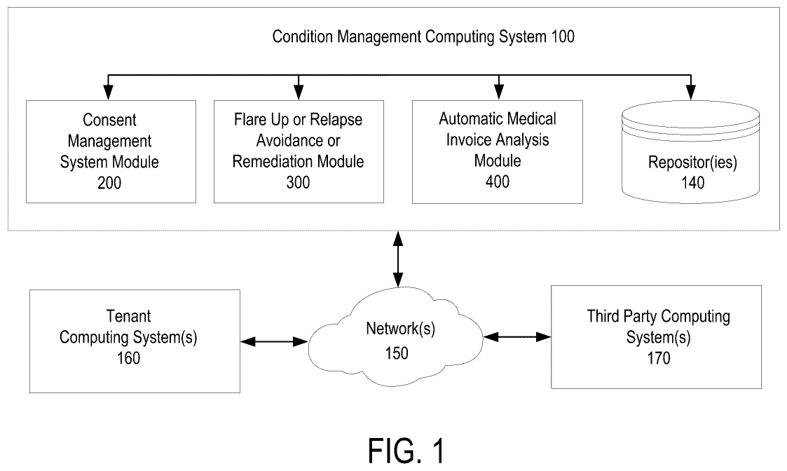

FIG. 1 depicts an example of a computing environment, according to various aspects, that can be used for (1) securely managing consents to take action to assist an individual in managing a health-related condition and/or in managing payments for treatments related to managing such a condition; (2) managing and storing an individual's healthcare-related data while also assuring compliance with one or more privacy laws or standards, such as HIPPA; and (3) predicting relapses and/or flare-ups in an individual's condition and, in response, automatically initiating an action via a third party computing system to prevent or treat the predicted relapse or flare-up.

A condition management computing system 100 may be provided that includes software components and/or hardware components for: (1) securely managing consents to take action to assist an individual in managing a health-related condition and/or in managing payments for treatments related to managing such a condition; (2) managing and storing an individual's healthcare-related data while also assuring compliance with one or more privacy laws or standards, such as HIPPA; and/or (3) predicting relapses and/or flare-ups in an individual's condition and, in response, automatically initiating an action via a third party computing system to prevent or treat the predicted relapse or flare-up. It should be understood that the condition management computing system 100 may provide a service that is accessible over one or more networks 150 (e.g., the Internet) by an entity (e.g., a tenant computing system 160 associated with the entity). Here, personnel of the entity may access the service over the one or more networks 150 through one or more graphical user interfaces (e.g., webpages). In addition, the condition management computing system 100 may include one or more interfaces (e.g., application programming interfaces (APIs)) for communicating with, accessing, and/or analyzing one or more third party computing system(s) 170 over the network(s) 150. For example, the condition management computing system 100 may access a third party computing system 170 for the entity via one of the interfaces to, for example: (1) retrieve information from or transmit information to the third-party computing system; (2) coordinate one or more services to be provided via the third party computing system 170; and/or (3) facilitate any other such activity (e.g., service, data exchange, order, etc.) discussed herein. Example services provided by third parties may include, for example, logistics services such as food delivery services, transportation services, insurance-related services (or other financial services), medical care, or any other suitable service.

In various embodiments, example third party computing systems 170 include, for example, one or more computing systems of: (1) health care providers (physical or mental health providers); (2) insurance providers; (3) pharmaceutical manufacturers; (4) pharmacies; (5) online marketplaces, such as Amazon.com or Walmart.com; (6) transportation providers, such as one or more taxi services or ride sourcing services (e.g., Uber or Lyft); (7) public transportation services; (8) fresh prepared food delivery services (e.g., Uber Eats or Door Dash); (9) grocery delivery services; (10) pharmacy delivery services; or (11) any other suitable third party system.

In some instances, the condition management computing system 100 may include one or more data repositories 140 that can be used for storing various types of data including patient identification data, electronic medical records, and/or any other suitable data that may be useful in facilitating various aspects of the inventions described herein. The system may be configured to receive data for storage within the one or more repositories 140 in any of our variety of different ways. For example, the system may be adapted to receive such data via: (1) one or more electronic medical records; (2) direct input from the individual (e.g., via a suitable user interface of a computing device); (3) one or more cameras (e.g., the camera of the individual's computer or mobile computing device); (4) one or more wearable computing devices worn by the user (e.g., a smart watch, an electronic step counter, an electronic heart rate sensor, etc.); (5) one or more handheld computing devices (e.g., the user's cell phone or tablet computing device); (6) one or more medical peripheral devices, such as an electronic scale, an electronic blood pressure reading device, a pulse oximeter, or the like; or (7) any other suitable data source.

According to various aspects of the disclosure, the condition management computing system 100 may comprise computing hardware performing a number of different processes in conjunction with, for example: (1) securely managing consents to take action to assist an individual in managing a health-related condition and/or in managing payments for treatments related to managing such a condition; (2) managing and storing an individual's healthcare-related data while also assuring compliance with one or more privacy laws or standards, such as HIPPA; and (3) predicting relapses and or flare-ups in an individual's condition and, in response, automatically initiating an action via a third party computing system to prevent or treat the predicted relapse or flare-up. For instance, the condition management computing system 100 according to various aspects executes a content management system module 110 in order to manage one or more patient consents needed to execute one or more aspects of the invention.

The condition management computing system 100 may also include a flare-up or relapse avoidance or remediation module 115. In various aspects, the condition management computing system 100 executes this module to predict the potential occurrence of a flare-up or relapse in a particular individual of a particular medical condition.

The condition management computing system 100 may also include an automatic medical invoice analysis module 120. In various aspects, the condition management computing system 100 executes this module 120 to analyze one or more medical invoices to determine whether any billing errors exist within the invoices.

### Data Input and Management

In particular embodiments, the system may be adapted to serve as a consolidated repository and data management system for a wide variety of information that may be used to support an individual in managing one or more medical conditions. Such information may include for example: (1) the individual's medical information; (2) information regarding the individual's diet and exercise regimen; (3) information regarding the individual's food intake and exercise history; (4) financial information that is relevant to the individual's treatment of their condition; (5) consent information that is required, for example, for the system to advocate or facilitate treatment on the individual's behalf; and (6) educational information that the individual may use in conjunction with their condition; and/or any other suitable information.

Particular examples of information that the system may collect and store include, for example: (1) symptoms that the individual is currently experiencing; (2) information regarding the individual's state of mind; (3) the amount and type of exercise that the individual completes; (4) information regarding the medications that the individual has taken; (5) information regarding any physical therapy at the individual has completed; (6) dietary information (information regarding the specific food and liquids that the individual has consumed); (7) information regarding bowel movements; (8) information regarding the individual's menstrual cycle; (9) information from a peripheral device that analyzes the individual's blood or urine), etc. Additional examples include the following types of information for the individual: (1) particular changes in the individual's weight; (2) one or more particular states of mind of the individual (e.g., on a particular day or over a particular span of time); (3) compliance or lack of compliance with a particular drug regimen; (4) compliance or a lack of compliance with a dietary program; (5) intake of particular foods or beverages; (6) exposure to particular environments, such as smoky or otherwise polluted environments; (7) compliance or lack of compliance with a particular exercise program; (8) the shape or structure of the individual's bowel movements; (9) specific properties of the individual's blood or urine, or other factors related to the individual's health or habits. In particular embodiments, the system may collect and store such information substantially in real time so the system can track the individual's health substantially in real time. In alternative embodiments, the system may collect and store the information in any appropriate alternative cadence.

In various embodiments, the system may also take, and store to memory, a picture of the individual on a daily basis (e.g., a picture of the individual's face or other body parts and store these images as part of the individuals electronic medical record), or any other suitable frequency. The system may also coordinate the intake of pictures of the individual's bowel movements. In particular embodiments, the system collects this information on an ongoing basis and stores this information in memory. This may allow the system to collect and maintain a detailed historical record of the individual's medical information that can be studied and mined using big data/machine learning computer processing techniques and/or rules-based computing techniques to identify trends over time that may be used to facilitate and enhance the individual's medical treatment.

### Consent Management Module

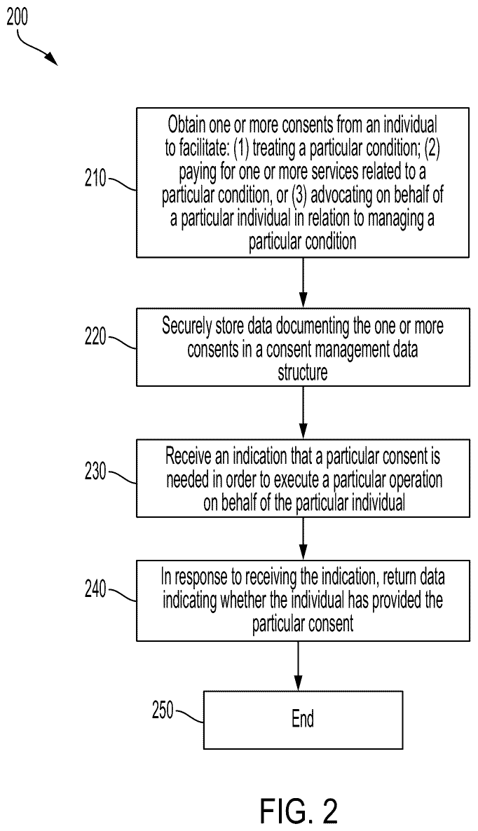

As noted above, in particular embodiments, in order to facilitate various aspects of the system described above, the system may include a consent management system for managing obtaining relevant consents from the individual and for making sure that the system has the necessary consents in place before taking corresponding action on behalf of the individual. FIG. 2 depicts an example of a process performed by an example consent management system module 200. This process includes operations that the condition management computing system 100 may execute in managing one or more patient consents needed in executing one or more steps described in this disclosure.

In this example, the system begins at step 210 where it obtains one or more consents from an individual to facilitate: (1) accessing the individual's medical data (e.g., from one or more electronic medical records or other sources); (2) using the individual's medical data to identify one or more suitable health care providers that are qualified to treat the individual for one or more conditions identified in the individual's medical data or in other sources; (3) paying for one or more goods or services on behalf of the client (e.g., those needed to treat the individual's particular condition and/or one or more symptoms associated with the individual's particular condition); and/or (4) advocating on behalf of a particular individual in relation to managing a particular condition (e.g., advocating on behalf of an insurance company or other party to facilitate coverage, one or more payments, or one or more discounts for goods or services related to treating the individual's condition).

Such consents may further include, for example: (1) consent for the system and/or an entity associated with the system to advocate on the individual's behalf with the individual's insurance company; (2) consent for the system to share the individual's medical data with one or more medical providers or other individuals such as one or more of the individual's relatives, caregivers, or patient advocates; (3) consent for the system to pay for one or more services or items from one or more payment sources specified by the individual-such payment sources may include, for example, one or more credit cards, one or more bank accounts, or other funding sources; and/or (4) consent for the system to use one or more logins for one or more apps or programs associated with the individual to order goods or services through those apps or programs (for example, this consent may include authorization for the system to order items from the individual's Amazon.com account or to order transportation through the individual's Uber account). The system may obtain such consents, for example, by prompting the user for input via a suitable computer interface, such as a suitable graphical user interface, voice interface, head set or any other suitable interface.

Next, the system advances to step 220, where it stores data documenting the one or more consents in a suitable consent management data structure. Such a data structure may include, for example, one or more databases or any other suitable data structure. Later, at step 230, the system receives an indication that a particular consent is needed in order to execute a particular operation on behalf of the particular individual. Next, at step 240, in response to receiving this indication, the system returns data indicating whether the individual has provided the particular consent. Finally, the system completes execution of the module at step 250.

For example, if an individual had provided consent at step 210 to charge the individual's credit card for any items that the system has determined to be necessary for the individual's treatment, and the system has determined that the individual needs one or more particular items to assist in the individual's treatment, the system would, at steps 230 and 240, first query the database to determine whether adequate consent has been given to both purchase the items and coordinate delivery of the items to the individual. In response to determining that the individual has provided the required consent, the system may, for example, automatically order the items to be delivered to the individual (e.g., through a particular online marketplace such as Amazon.com.) Other particular examples include: (1) receiving an individual's consent to schedule appointments on behalf of the individual and, in response, scheduling those appointments; (2) receiving an individual's consent to schedule transportation on behalf of the individual and, in response, coordinating transportation of the individual, for example, to a medical appointment; (3) receiving an individual's consent to order and pay for medication on behalf of the individual and/or to have the medication delivered to the individual and, in response, purchasing the medication for the individual and having the medication delivered to the individual, for example, at the individual's home.

### Healthcare Provider Rating, Recommendation and Scheduling, and Rating and Delivery of Goods

In various embodiments, the system may be adapted to facilitate reviews (e.g., online reviews) of relevant goods and service providers and to store the results of such reviews in system memory. In particular embodiments, the system may be adapted to allow users to search for relevant goods and service providers (e.g., via a suitable user interface, such as a graphical user interface) and, in response to a particular search identifying a particular good, or service provider, displaying or otherwise communicating reviews and/or one or more ratings for the service provider or good to the user.

The system may also be configured to automatically identify one or more service providers or goods for the individual. In particular embodiments, the system may be configured to automatically purchase and schedule the delivery of such identified goods (e.g., via a suitable online marketplace). Such goods may include, for example, one or more kits of goods that have been specifically assembled together to treat the individual's condition and/or to assist the individual before, during, or after a particular procedure. For example, the kit may be a post-surgical supply kit that includes a plurality of items that have been selected to assist an individual after a particular surgery.

The system may also or alternatively be configured to automatically schedule one or more appointments with such identified service providers. In various embodiments, when making such scheduling, the system may be configured to automatically add one or more entries to one or more electronic calendars associated with the individual to indicate when the appointment will occur and/or when the scheduled transportation to the appointment will happen. Similarly, when scheduling the delivery of items such as groceries or other items to the individual, the system may also automatically add one or more entries to one or more electronic calendars associated with the individual to indicate that such items are to be delivered and/or when such items are to be delivered.

### Coordination of Transportation

In various embodiments, the system may be adapted to automatically coordinate transportation for that particular individual. For example, in certain circumstances, the system may automatically arrange for the individual to be picked up from a particular location at a particular time (e.g., by a particular taxi service or ride-sourcing service such as Uber or Lyft) and taken to a particular target location such as a healthcare provider's office. In situations where the system has obtained and/or stored the individual's consents to coordinate such transportation and/or to pay for such transportation on the individual's behalf (e.g., using a payment method provided by an associated with the individual), the system may coordinate and pay for such transportation automatically and without input from the individual. More particular examples and use cases are discussed in more detail below.

### Financial Advocacy

In various embodiments, the system may be further adapted to assist an individual in managing various financial aspects of managing their condition. For example, the system may be adapted to automatically assist an individual in identifying and obtaining available discounts on one or more drugs associated with the individual's condition. This may include, for example, identifying one or more coupons associated with the drugs, or identifying one or more discount or patient assistance program through which the one or more drugs are available at a discounted rate.

As another example, the system may be adapted to automatically facilitate advocating for and/or obtaining one or more insurance pre-authorizations for one or more medications, medical items, medical services, and/or procedures that are needed for the treatment of the individual's condition. In various embodiments, if the system has obtained and/or stored sufficient user consent to do so, the system may use one or more form letters, and/or AI chatbot technology to advocate directly with the insurance company on the individual's behalf to obtain such pre-authorizations.

As a further example, the system may be adapted to automatically identify one or more suitable patient advocacy or assistance programs that may, for example, provide financial assistance to individuals that have difficulty affording treatments associated with the individual's condition. Such advocacy or assistance programs may include those provided by pharmaceutical companies, public interest groups, non-profit organizations, and/or any other suitable entity.

After identifying one or more such groups that may be willing to provide assistance to the individual, the system may, for example, automatically request assistance from the one or more identified groups on behalf of the individual. Alternatively, or in addition, the system may automatically notify the individual of the particular group and/or inform the individual of the potential assistance that the groups may be willing to provide.

As another example, the system may be adapted to automatically review one or more financial transactions associated with one or more goods or services associated with treating the individual's condition to identify any potential billing errors associated with the one or more financial transactions. In response to identifying the one or more billing errors, the system may automatically initiate action to resolve those errors. For example, the system may automatically generate an electronic communication to an entity associated with the relevant financial transaction identifying the financial transaction, the individual, and/or the identified billing error. The system may, alternatively, or in addition, generate such a communication to the individual so that the individual may be aware of the billing error and/or take action to correct the billing error.

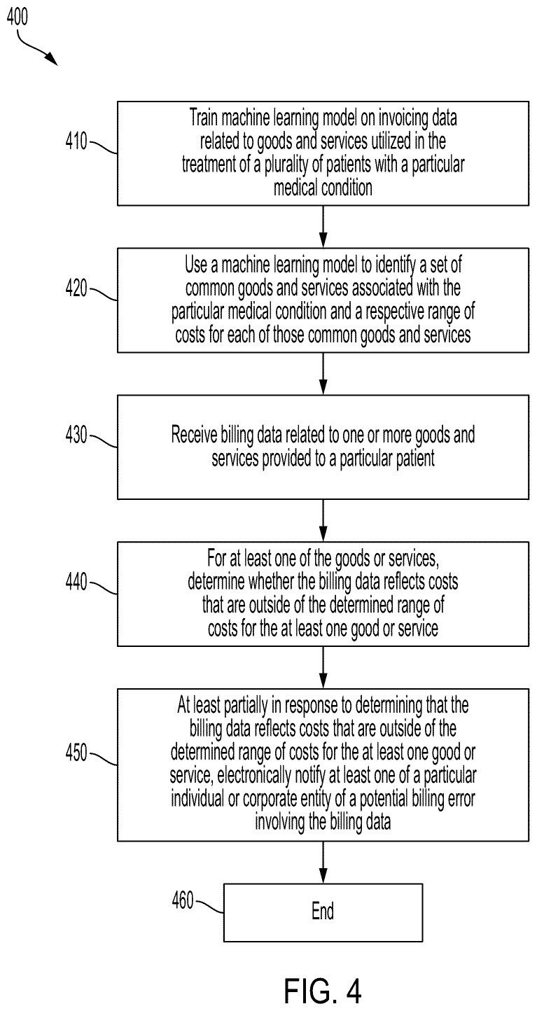

In doing so, the system may execute the automatic medical invoice analysis module 400, which is shown in FIG. 4. When executing this module, the system begins at step 410, where the system trains a machine learning model on invoicing data related to goods and services utilized in the treatment of a plurality of individuals with a particular medical condition. Next, the system advances to step 420, where it uses a machine learning model to identify a set of common goods and services associated with the particular medical condition and a respective range of costs for each of those common goods and services.

Next, at step 430, the system receives billing data related to one or more goods and services provided to a particular individual. The system then advances to step 440, where, for at least one of the goods or services, the system determines whether the billing data reflects costs that are outside of the determined range of costs for the at least one good or service. In various embodiments, in response to determining that the billing data reflects costs that are outside of the predetermined range of costs for the at least one good or service, the system, at step 450, electronically notifies (e.g., via a suitable electronic message or other suitable communication) at least one particular individual or corporate entity of a potential billing error involving the billing data. Finally, at step 460, the system ends execution of the module.

Diet, Medical and/or Exercise Regimen Compliance

As noted above, in various embodiments, the system may store a diet, medical, and/or exercise regimen for the user in system memory. In addition, or alternatively, the system may store information regarding the individual's compliance with one or more diet, medical, and/or exercise regimens for the user including such regimens that are stored in system memory. The system may use this information to determine whether the individual is complying with one or more diet, medical, and/or exercise regimens. If so, the system may be adapted to communicate positive feedback to the user in the form of one or more positive messages, badges, etc. The system may also alternatively communicate information regarding the individual's compliance with the one or more regimens to one or more third parties, such as the individual's health care provider(s), relative(s), or the individual's friends and/or followers (or the general public) via social media.

In response to the individual not complying with the one or more diet, medical, and/or exercise regimens, the system may, for example, communicate (e.g., display) one or more coaching messages to the individual informing the user of their deficiency in complying with the one or more regimens, and encouraging the individual to take action to comply. As discussed in greater detail below, the system may also use this evidence of non-compliance with one or more regimens as a factor in assessing the individual's risk of suffering a flare-up or relapse in their condition.

### Patient Education-Condition and Related Medications

As noted above, in particular embodiments, the system may store information regarding an individual's condition and/or one or more medications used to treat the individual's condition. The system may, for example, periodically display one or more messages to the user to help educate the user regarding their condition. In particular embodiments, the system may display one or more of such messages in response to one or more triggers, such as the user not complying with one or more healthcare, dietary or exercise regimens, or simply at a time when the system determines that the individual is most likely to read the message. In particular embodiments, the system may be further or alternatively configured to allow the user to search for answers to questions regarding their condition and/or related medications, e.g., using one or more electronic search tools.

### Tracking Delivery of Drugs or Other Items

In various embodiments, the system may be adapted track the delivery of drugs or other items that are to be delivered to the patient or other individual and to display or otherwise communicate relevant information regarding the delivery to the patient. For example, in various embodiments, the system may track the location of the drugs or other items as the drugs or other items are being delivered from a point of origin to the patient. In particular embodiments, the system may communicate the location of the drugs or other items to the patient substantially in real time, so that the patient can plan their schedule accordingly and/or quickly identify any potential delays in the delivery.

### Flare-up or Relapse Avoidance or Remediation

As discussed above, the system may be configured to use machine learning and/or big data processing techniques to assess the likelihood that the individual will suffer a flare-up or relapse in their condition in the near future (e.g., within a pre-determined number of days). The system may then take steps to avoid or treat the flare-up or relapse.

In this regard, in particular embodiments, the system may collect a large amount of data regarding an individual through a plurality of different sources. These sources may include, for example: (1) manual input by the user; (2) the user's electronic medical records; (3) information gathered by wearable devices (e.g., one or more smart watches or step trackers): (4) medical peripheral devices such as blood pressure tracking devices and/or electronic scales; and/or (5) information gathered by the individual's smartphone. Such information may include, for example: (1) symptoms that the individual is currently experiencing; (2) information regarding the individual's state of mind; (3) the amount and type of exercise that the individual completes; (4) information regarding the medications that the individual has taken; (5) information regarding any physical therapy at the individual has completed; (6) dietary information (e.g., information regarding the specific food and liquids that the individual has consumed); (7) information regarding bowel movements; (8) information regarding the individual's menstrual cycle; (9) information from a peripheral device that analyzes the individual's blood or urine), etc. As noted above, the system may obtain and store this data substantially in real time and on a substantially ongoing basis so that the system has sufficient data to track the individual's health substantially in real time and/or on an ongoing basis.

In various embodiments, the system may also take a picture of the individual on a daily basis (e.g., a picture of the individual's face or one or more other body parts and store these images as part of the individual's electronic medical record). The system may also coordinate the intake of one or more pictures of the individual's bowel movements. In particular embodiments, the system collects this information on an ongoing basis and stores this information in memory. This may allow the system to collect and maintain a detailed historical record of the individual's medical information that can be studied and mined using big data and/or machine learning data processing techniques to identify one or more trends over time that may be used to facilitate and enhance the individual's medical treatment.

In particular embodiments, the system may gather such data for a plurality of individuals (e.g., a large number of individuals) having the same condition. This may allow the system to use big data/machine learning techniques to identify one or more trends associated with the condition. For example, the system may identify one or more factors that are associated with a flare-up or relapse in the client's condition.

As a particular example, the system may determine that one or more of the following factors, alone or in combination, are commonly associated with a flare-up or relapse in a particular condition: (1) particular changes in an individual's weight; (2) one or more particular states of mind (e.g., on a particular day or over a particular span of time); (3) compliance or lack of compliance with a particular drug regimen; (4) compliance or a lack of compliance with a particular dietary program; (5) intake of particular foods or beverages; (6) exposure to particular environments, such as smoky or otherwise polluted environments; (7) compliance or lack of compliance with a particular exercise program; (8) the shape or structure of the individual's bowel movements; and/or (9) specific properties of the individual's blood or urine, or other factors.

The system may make such determinations for the individual alone and/or for a group of individuals with the same or similar condition. In particular embodiments, the system may determine a risk assessment indicator (such as a percentage) that indicates how frequently the identified one or more factors are actually associated with a particular flare-up or relapse in the individual's condition. For example, the system may determine that 50% of the time when a particular individual consumes dairy products and hasn't taken a particular medication within the past 24 hours, the individual sufferers a flair-up or relapse in their condition. As another example, the system may determine that, on average, 40% of the time when a particular individual within a particular group of individuals has been depressed for over a week, has consumed more than a particular threshold number of alcoholic beverages within that week, and has gained over a particular percentage of their body weight within that week, they suffer a flare-up or a relapse in their condition sometime within the following week (or any other specified period of time).

In particular embodiments, the system may take action in response to determining that the risk of a particular individual suffering a flare-up or relapse exceeds a particular threshold value. For example, if the system determines that, at a particular time, the particular individual has over a 70% chance of suffering a relapse or flare-up in the following week, the system may decide to take one or more actions to prevent or reduce the severity of the predicted potential flare-up or relapse.

For example, the system may display a message to the individual indicating that they are at particular risk of suffering a potential flare-up or relapse and provide instructions to the individual for measures that they may take to potentially avoid the flare-up or relapse. As a particular example, if the system determines that the individual is at risk of suffering a relapse or a flare-up because they haven't taken a particular medicine, the system may automatically convey a message to the user indicating that they should take their medicine.

As an additional example, in response to determining that a particular individual has over a threshold chance of suffering a relapse or flare-up in the following week, the system may initiate electronic communication with a third-party computing system (e.g., via a suitable application programming interface (API)), and, through that communication, initiate one or more actions that are intended to avoid or treat the anticipated relapse or flare-up. For example, the system may automatically order one or more days' worth of groceries to be delivered to the individual's home. In particular embodiments, the system may have automatically contacted a dietary professional or referenced a database containing suitable dietary information and used information from that contact to select groceries that are intended to avoid or treat the anticipated relapse or flare-up. In this example, the groceries to be delivered to the individual's home may, for example, include those selected groceries.

As a further example, in response to determining that the particular individual has over a threshold chance of suffering a relapse or flare-up in the following week, the system may automatically schedule an appointment (e.g., either an in-person or virtual appointment), such as an appointment with an appropriate physician, mental health professional, physical therapist, or other service provider. In particular embodiments, the system may automatically select the service provider for the appointment based, for example, on one or more symptoms that the individual is anticipated to experience during the predicted flare-up or relapse. For example, the system may automatically search one or more reviews of a plurality of relevant service providers and, based at least in part on the reviews from other customers of the service provider, select a provider that is indicated by the reviews to have experience trading flare-ups or relapses of the particular condition and/or symptoms of such flare-ups or relapses.

In various embodiments, the system may also coordinate transportation for the individual (e.g., through a particular ride-sourcing service) to the relevant appointment. For example, the system may automatically coordinate transportation for the individual to and from an appointment with the particular medical provider via a suitable ride-sourcing service such as Uber or Lyft.

In various embodiments, when making such scheduling, the system may be configured to automatically add one or more entries to one or more electronic calendars associated with the individual to indicate when the appointment will occur and/or when the scheduled transportation to the appointment will happen. Similarly, when scheduling the delivery of items such as groceries or other items to the individual, the system may also automatically add one or more entries to one or more electronic calendars associated with the individual to indicate that such items are to be delivered and/or when such items are to be delivered.

In particular embodiments, the system may coordinate the delivery of one or more kits of items that have been assembled to treat the predicted flare-ups or relapses. For example, the kit may include two or more particular medical supplies that have been assembled together into a kit for purposes of treating such flare-ups or relapses.

Furthermore, in particular embodiments, the system may be adapted to, in response to predicting that an individual may suffer that particular flare-up or relapse, automatically notify one or more physicians or other individuals associated with the particular individual. Such other individuals may include, for example, the individual's spouse, parent(s), or other caregivers or patient advocates.

The system may be further adapted to, in response to predicting that an individual may suffer a particular flare-up or relapse, automatically initiate a request for one or more insurance pre-authorizations for one or more healthcare visits or procedures needed to address the predicted flare-up or relapse. This may, for example, eliminate delays in the individual obtaining treatment if they suffer the anticipated flare-up or relapse. In various embodiments, the system may obtain suitable pre authorizations for particular appointments before scheduling such appointments and/or corresponding transportation as discussed above.

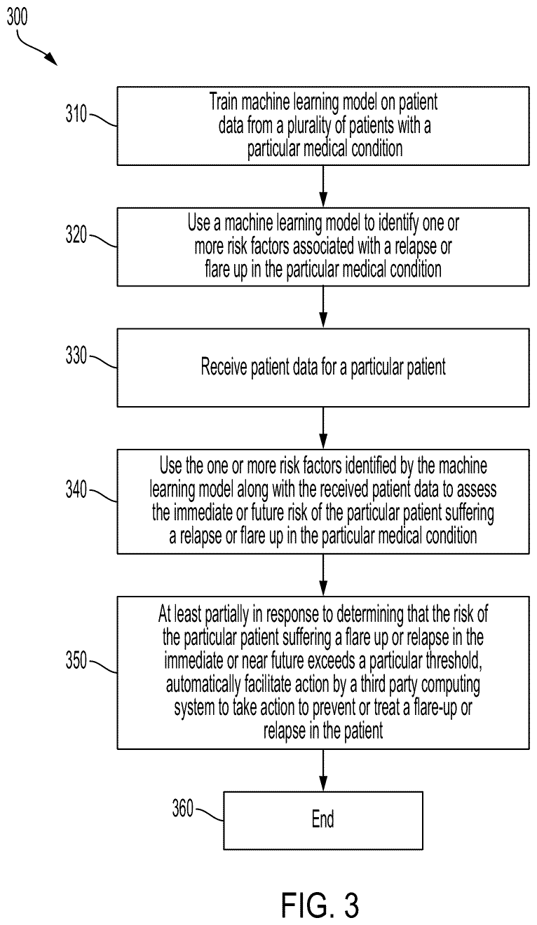

FIG. 3 more generally outlines the steps performed by a flare-up or relapse avoidance or remediation module 300 according to various embodiments. As may be understood from this figure, when executing this module, the system begins at step 310, where it trains a machine learning model on patient data from a plurality of individuals with a particular medical condition. Next, and step 320, the system uses the machine learning model to identify one or more risk factors associated with a relapse or flare-up in the particular medical condition. Later, at step 330, the system receives patient data for a particular individual (e.g., by retrieving the information from a suitable data repository, such as an internal or external data repository). Such data may include, for example, any of the data collected by the system in regard to the individual as discussed above and/or any other suitable information.

Next, at step 340, the system uses the one or more risk factors identified by the machine learning model along with the received patient data to assess the immediate or future risk of the particular individual suffering a relapse or flare-up in the particular medical condition. Finally, at step 350, at least partially in response to determining that the risk of the particular individual suffering a flare-up or relapse in the immediate or near future exceeds a particular threshold, the system automatically facilitates action by a third-party computing system to take action to prevent or treat a flare-up or relapse in the individual.

As discussed in more detail above, such action may include, for example: (1) coordinating the purchase and/or delivery of one or more medications or other items to the individual; (2) identifying a suitable healthcare provider (physical or mental healthcare provider) to provide treatment for the individual's anticipated flare-up or relapse; (3) making one or more appointments for the individual with one or more healthcare providers or other service providers to avoid the anticipated flare-up or relapse, or to treat the anticipated flare-up or relapse; (4) advocating for and/or obtaining one or more insurance pre-authorizations that are required for one or more services associated with the individual's condition-such services maybe services that are specifically directed to avoiding or treating the individual's anticipated flare-up or relapse; and/or (5) coordinating one or more transportation services on the individual's behalf as discussed above.

### Example Technical Platforms

Aspects of the present disclosure may be implemented in various ways, including as computer program products that include articles of manufacture. Such computer program products may include one or more software components including, for example, software objects, methods, data structures, and/or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query, or search language, and/or a report writing language. In one or more example aspects, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established, or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

According to various aspects, a non-volatile computer-readable storage medium may include a floppy disk, flexible disk, hard disk, solid-state storage (SSS) (e.g., a solid-state drive (SSD), solid state card (SSC), solid state module (SSM)), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

According to various aspects, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where various aspects are described to use a computer-readable storage medium, other types of computer-readable storage media may be substituted for or used in addition to the computer-readable storage media described above.

Various aspects of the present disclosure may also be implemented as methods, apparatuses, systems, computing devices, computing entities, and/or the like. As such, various aspects of the present disclosure may take the form of a data structure, apparatus, system, computing device, computing entity, and/or the like executing instructions stored on a computer-readable storage medium to perform certain steps or operations. Thus, various aspects of the present disclosure also may take the form of entirely hardware, entirely computer program product, and/or a combination of computer program product and hardware performing certain steps or operations.

Various aspects of the present disclosure are described below with reference to block diagrams and flowchart illustrations. Thus, each block of the block diagrams and flowchart illustrations may be implemented in the form of a computer program product, an entirely hardware aspect, a combination of hardware and computer program products, and/or apparatuses, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some examples of aspects, retrieval, loading, and/or execution may be performed in parallel such that multiple instructions are retrieved, loaded, and/or executed together. Thus, such aspects can produce specially configured machines performing the steps or operations specified in the block diagrams and flowchart illustrations. Accordingly, the block diagrams and flowchart illustrations support various combinations of aspects for performing the specified instructions, operations, or steps.

### Example System Architecture

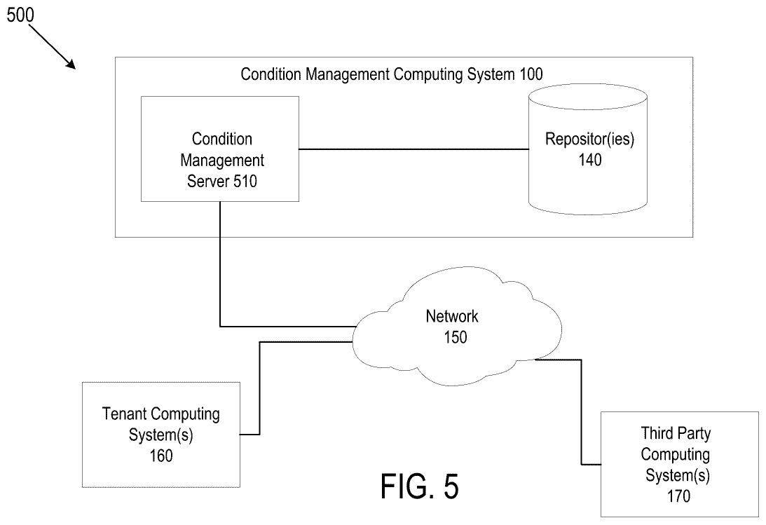

FIG. 5 is a block diagram of a system architecture 500 that can be used in a Condition Management Computing System 100 according to various aspects of the disclosure as detailed herein. Components of the system architecture 500 are configured according to various aspects to facilitate one or more aspects of the system's functionality as described above. As may be understood from FIG. 5, the system architecture 500 according to various aspects may include a Condition Management Computing system 100 that comprises one or more condition management servers 510 and one or more data repositories 140 that may, for example, store patient data such as the data described above, and other data needed to execute one or more additional aspects and the systems functionality. Although the condition management server(s) 510 and repository (ies) 140 are shown as separate components, it should be understood that according to other aspects, these components 510, 140 may comprise a single server and/or repository, a plurality of servers and/or repositories, one or more cloud-based servers and/or repositories, or be in any other suitable configuration.

The condition management server(s) 510 may communicate, access, analyze, and/or the like one or more tenant computing systems 160 and/or one or more third party computing systems 170 of over one or more networks 150 and may execute a consent management system module 110, flare-up or relapse avoidance or remediation module 300, and/or automatic medical invoice analysis module 400 as described herein. In addition, according to particular aspects, the condition management server(s) 510 provide one or more graphical user interfaces and other interfaces through which personnel of the entities (via the tenant computing system(s) 160) interact with the condition management computing system 100, as well as allowing the condition management computing system 100 to communicate with the third party computing system(s) 170. Thus, the condition management server(s) 510 may interface with the third-party computing system(s) 170 via one or more suitable application programming interfaces (APIs), direct connections, and/or the like.

### Example Computing Hardware

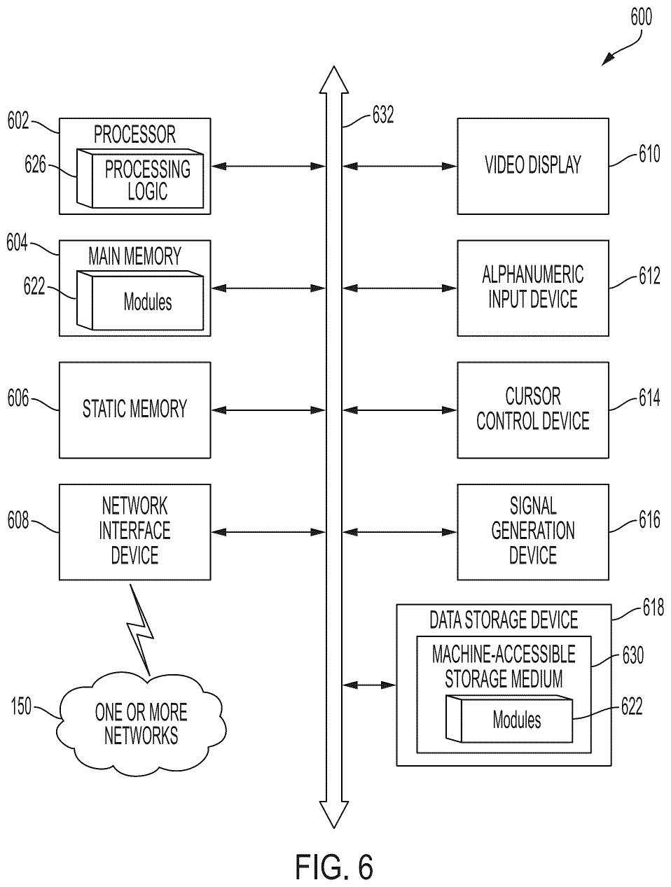

FIG. 6 illustrates a diagrammatic representation of a computing hardware device 600 that may be used in accordance with various aspects of the disclosure. For example, the hardware device 600 may be computing hardware such as a condition management server 510 as described in FIG. 5. According to particular aspects, the hardware device 600 may be connected (e.g., networked) to one or more other computing entities, storage devices, and/or the like via one or more networks such as, for example, a LAN, an intranet, an extranet, and/or the Internet. As noted above, the hardware device 600 may operate in the capacity of a server and/or a client device in a client-server network environment, or as a peer computing device in a peer-to-peer (or distributed) network environment. According to various aspects, the hardware device 600 may be a personal computer (PC), a tablet PC, a set-top box (STB), a Personal Digital Assistant (PDA), a mobile device (smartphone), a web appliance, a server, a network router, a switch or bridge, or any other device capable of executing a set of instructions (sequential or otherwise) that specify actions to be taken by that device. Further, while only a single hardware device 600 is illustrated, the term “hardware device,” “computing hardware,” and/or the like shall also be taken to include any collection of computing entities that individually or jointly execute a set (or multiple sets) of instructions to perform any one or more of the methodologies discussed herein.

The hardware device 600 includes a processor 602, a main memory 604 (e.g., read-only memory (ROM), flash memory, dynamic random-access memory (DRAM) such as synchronous DRAM (SDRAM), Rambus DRAM (RDRAM), and/or the like), a static memory 606 (e.g., flash memory, static random-access memory (SRAM), and/or the like), and a data storage device 618, that communicate with each other via a bus 632.

The processor 602 may represent one or more general-purpose processing devices such as a microprocessor, a central processing unit, and/or the like. According to some aspects, the processor 602 may be a complex instruction set computing (CISC) microprocessor, reduced instruction set computing (RISC) microprocessor, very long instruction word (VLIW) microprocessor, a processor implementing other instruction sets, processors implementing a combination of instruction sets, and/or the like. According to some aspects, the processor 602 may be one or more special-purpose processing devices such as an application specific integrated circuit (ASIC), a field programmable gate array (FPGA), a digital signal processor (DSP), network processor, and/or the like. The processor 602 can execute processing logic 626 for performing various operations and/or steps described herein.

The hardware device 600 may further include a network interface device 808, as well as a video display unit 610 (e.g., a liquid crystal display (LCD), a cathode ray tube (CRT), and/or the like), an alphanumeric input device 612 (e.g., a keyboard), a cursor control device 614 (e.g., a mouse, a trackpad), and/or a signal generation device 616 (e.g., a speaker). The hardware device 600 may further include a data storage device 618. The data storage device 618 may include a non-transitory computer-readable storage medium 630 (also known as a non-transitory computer-readable storage medium or a non-transitory computer-readable medium) on which is stored one or more modules 622 (e.g., sets of software instructions) embodying any one or more of the methodologies or functions described herein. For instance, according to particular aspects, the modules 622 include the consent management system module 200, the flare-up or relapse avoidance or remediation module 300, and the automatic medical invoice analysis module 500 as described herein. The one or more modules 622 may also reside, completely or at least partially, within main memory 604 and/or within the processor 602 during execution thereof by the hardware device 600—main memory 604 and processor 602 also constituting computer-accessible storage media. The one or more modules 622 may further be transmitted or received over a network 150 via the network interface device 608.

While the computer-readable storage medium 630 is shown to be a single medium, the terms “computer-readable storage medium” and “machine-accessible storage medium” should be understood to include a single medium or multiple media (e.g., a centralized or distributed database, and/or associated caches and servers) that store the one or more sets of instructions. The term “computer-readable storage medium” should also be understood to include any medium that is capable of storing, encoding, and/or carrying a set of instructions for execution by the hardware device 600 and that causes the hardware device 600 to perform any one or more of the methodologies of the present disclosure. The term “computer-readable storage medium” should accordingly be understood to include, but not be limited to, solid-state memories, optical and magnetic media, and/or the like.

### System Operation

The logical operations described herein may be implemented (1) as a sequence of computer implemented acts or one or more program modules running on a computing system and/or (2) as interconnected machine logic circuits or circuit modules within the computing system. The implementation is a matter of choice dependent on the performance and other requirements of the computing system. Accordingly, the logical operations described herein are referred to variously as states, operations, steps, structural devices, acts, or modules. These states, operations, steps, structural devices, acts, and modules may be implemented in software, in firmware, in special purpose digital logic, and any combination thereof. Greater or fewer operations may be performed than shown in the figures and described herein. These operations also may be performed in a different order than those described herein.

It should be understood that the system may include any suitable user interfaces. Such user interfaces may include visual interfaces, verbal interfaces (e.g., one or more artificial intelligence Chatbots such as ChatGPT), or any other suitable interface. In a particular embodiment, the system uses an AI Chatbot as the primary interface with the user.

### CONCLUSION

While this specification contains many specific aspect details, these should not be construed as limitations on the scope of any invention or of what may be claimed, but rather as descriptions of features that may be specific to particular aspects of particular inventions. Certain features that are described in this specification in the context of separate aspects also may be implemented in combination in a single aspect. Conversely, various features that are described in the context of a single aspect also may be implemented in multiple aspects separately or in any suitable sub-combination. Moreover, although features may be described above as acting in certain combinations and even initially claimed as such, one or more features from a claimed combination may in some cases be excised from the combination, and the claimed combination may be a sub-combination or variation of a sub-combination.

Similarly, while operations are described in a particular order, this should not be understood as requiring that such operations be performed in the particular order described or in sequential order, or that all described operations be performed, to achieve desirable results. In certain circumstances, multitasking and parallel processing may be advantageous. Moreover, the separation of various components in the various aspects described above should not be understood as requiring such separation in all aspects, and the described program components (e.g., modules) and systems may be integrated together in a single software product or packaged into multiple software products.

Many modifications and other aspects of the disclosure will come to mind to one skilled in the art to which this disclosure pertains having the benefit of the teachings presented in the foregoing descriptions and the associated drawings. Therefore, it is to be understood that the disclosure is not to be limited to the specific aspects disclosed and that modifications and other aspects are intended to be included within the scope of the appended claims. Although specific terms are employed herein, they are used in a generic and descriptive sense only and not for the purposes of limitation. Additional information regarding various embodiments is included in Appendices A-D.

What is claimed is:

1. A system comprising:
a non-transitory computer-readable medium storing instructions; and
a processing device communicatively coupled to the non-transitory computer-readable medium, wherein the processing device is configured to execute the instructions and thereby perform operations comprising:
receiving a set of patient event data, the set of patient event data including at least one respective instance of a respective patient event experienced by each respective patient of a plurality of patients;
receiving a first set of parameters, the first set of parameters including a respective set of one or more parameters for each respective patient of the plurality of patients that were associated with the respective patient immediately before or while the patient experienced the at least one instance of the respective patient event;
receiving, for a particular patient, a set of particular patient parameters;
processing the first set of parameters, the set of patient event data, and the set of particular patient parameters using at least one of a rules-based model or a machine-learning model to generate a prediction of a future patient event for the particular patient;
receiving, for each respective patient event, respective event mitigation data;
receiving, for each respective patient event, respective event outcome data;
processing the prediction of the future patient event, the respective event mitigation data, and the respective event outcome data to generate a future patient event mitigation recommendation; and
facilitating implementation of a mitigating action defined by the future patient event mitigation recommendation.

2. The system of claim 1, wherein the step of processing the first set of parameters, the set of patient event data, and the set of particular patient parameters comprises:
identifying, for each respective patient event, based on a plurality of respective instances of the respective patient event, a subset of patient parameters that are commonly associated with the respective patient event, each of the subset of patient parameters being selected from the first set of parameters; and
processing the first set of parameters, the set of patient event data, the subset of patient parameters, and the set of particular patient parameters using at least one of a rules-based model or a machine-learning model to generate the prediction of a future patient event for the particular patient.

3. The system of claim 1, wherein the first set of parameters comprise one or more of a set of medication adherence parameters, a set of bowel movement parameters, a set of exercise parameters, a set of dietary parameters, a set of weight trend parameters, a set of psychological parameters, or a set of fitness tracking device parameters.

4. The system of claim 1, wherein the operations further comprise:
identifying a set of potentially mitigating actions for each respective patient event;
determining, for each respective potentially mitigating action from the set of potentially mitigating actions, a respective consent requirement;
generating a graphical user interface for soliciting consent, from the particular patient, for each respective potentially mitigating action by configuring the user interface by:
including a first set of interface elements that each correspond to a first subset of the set of potentially mitigating actions for which the respective consent requirement includes a requirement for affirmative consent, wherein each interface element of the first set of interface elements is configured to elicit, from the particular patient, the affirmative consent defined by the respective consent requirement for the respective potentially mitigating action; and
excluding a second set of interface elements that correspond to a second subset of the set of potentially mitigating actions for which the respective consent requirement does not include the requirement for affirmative consent; and
receiving, from the particular patient via the first set of interface elements on the graphical user interface, the affirmative consent for the first subset of potentially mitigating actions.

5. The system of claim 4, wherein the operations further comprise:
determining whether the particular patient has previously provided the affirmative consent for the mitigating action; and
in response to determining that the particular patient has previously provided the affirmative consent for the mitigating action, automatically facilitating implementation of the mitigating action without further prompting the particular patient for additional consent.

6. A computer-implemented data processing method for improving prediction and automated remediation of a future medical event, the method comprising:
receiving, by computing hardware, a set of patient parameters for a particular patient;
analyzing, by the computing hardware to produce a first data analysis result, the set of patient parameters using a machine-learning model trained with a set of patient parameter data derived from a plurality of patients, each of the plurality of patients having experienced a respective medical event from a set of medical events and having at least one respective parameter from the set of patient parameters;
generating, by the computing hardware based on the first data analysis result, a prediction as to an occurrence of the future medical event for the particular patient, the future medical event including at least one medical event from the set of medical events;
generating, by the computing hardware, a graphical user interface by configuring a display element that includes the prediction; and
providing, by the computing hardware, the graphical user interface for display on a user device.

7. The computer-implemented data processing method of claim 6, further comprising:
analyzing, by the computing hardware to produce a second data analysis result, the prediction and the set of patient parameters using a second machine-learning model trained with a set of patient event outcome data derived from the plurality of patients, the set of patient event outcome data including respective treatment data and respective outcome data for each of the plurality of patients;
generating, by the computing hardware based on the second data analysis result, a mitigation recommendation to improve an outcome for the future medical event for the particular patient; and
initiating, by the computing hardware, one or more processing operations or network communications according to the mitigation recommendation.

8. The computer-implemented data processing method of claim 7, wherein:
generating the graphical user interface further comprises configuring the graphical user interface to include a mitigation initiation control element configured to initiate the processing operations or the network communication according to the mitigation recommendation;
the method further comprises, receiving, by the computing hardware via the graphical user interface, selection of the mitigation initiation control element; and
initiating the one or more processing operations or network communications occurs in response to the selection of the mitigation initiation control element.

9. The computer-implemented data processing method of claim 7, wherein initiating the one or more processing operations or network communications according to the mitigation recommendation comprises at least one of:
initiating network communication with a third-party computing system to initiate logistics operations for delivering a particular set of products to the particular patient;
initiating processing operations to modify a frequency of notifications sent to the user device related to the set of patient parameters;
initiating network communication between the user device and a second computing device, the second computing device being identified based on the future medical event; or
generating a set of educational materials based on the future medical event and transmitting, via the network communication, the set of educational materials to the user device.

10. A computer-implemented data processing method comprising:
training, by computer hardware, a machine learning model on patient data from a plurality of patients with a particular medical condition;
using, by computer hardware, a machine learning model to identify one or more risk factors associated with a relapse or flare-up in the particular medical condition;
receiving, by computer hardware, patient data for a particular patient;
using, by computer hardware, the one or more risk factors identified by the machine learning model along with the received patient data to assess the immediate or future risk of the particular patient suffering a relapse or flare-up in the particular medical condition; and
at least partially in response to determining that the risk of the particular patient suffering a flare-up or relapse in the immediate or near future exceeds a particular threshold, automatically facilitating, by computer hardware, action by a third-party computing system to take action to prevent or treat a flare-up or relapse in the patient.

11. The computer-implemented data processing method of claim 10, wherein the particular medical condition is inflammatory bowel disease.

12. The computer-implemented data processing method of claim 10, wherein the action facilitated by the third-party computing system comprises facilitating the delivery of one or more food items to the patient.

13. The computer-implemented data processing method of claim 12, wherein the one or more food items comprise at least one day's worth of groceries for the patient.

14. The computer-implemented data processing method of claim 13, wherein computer-implemented data processing method comprises automatically accessing, by computer hardware, a database containing dietary information and using information identified from the database to select the one or more food items to prevent the particular patient from suffering a relapse or flare-up of the particular medical condition.

15. The computer-implemented data processing method of claim 14, wherein the particular medical condition is inflammatory bowel disease.

16. The computer-implemented data processing method of claim 10, wherein the action facilitated by the third-party computing system comprises scheduling an appointment with a service provider to perform one or more services for the patient.

17. The computer-implemented data processing method of claim 16, wherein the appointment is with a healthcare provider.

18. The computer-implemented data processing method of claim 17, wherein the computer-implemented data processing method comprises using computer hardware to automatically select the service provider for the appointment based, for example, on one or more symptoms that the individual is anticipated to experience during a flare-up or relapse of the particular medical condition.

19. The computer-implemented data processing method of claim 10, wherein the action facilitated by the third-party computing system comprises scheduling transportation for the patient via a ride-sourcing or taxi service.

20. The computer-implemented data processing method of claim 10, wherein the action facilitated by the third-party computing system comprises scheduling transportation for the patient via a ride-sourcing service to a medical appointment to treat or prevent the particular medical condition.
